(19) United States
(12) Patent Application Publication
(10) Pub. No.: US 2025/0041599 A1
(43) Pub. Date: Feb. 6, 2025

(54) DEVICES FOR PROVIDING ELECTROPHONIC MICROPHONE AND RELATED METHODS

(71) Applicants: Ohio State Innovation Foundation, Columbus, OH (US); Washington University, St. Louis, MO (US)

(72) Inventors: Oliver F. ADUNKA, Columbus, OH (US); Craig A. BUCHMAN, Clayton, MO (US); Amit WALIA, St. Louis, MO (US)

(21) Appl. No.: 18/717,314

(22) PCT Filed: Dec. 6, 2022
(86) PCT No.: PCT/US2022/052004

§ 371 Date: Jun. 6, 2024

Related U.S. Application Data
(60) Provisional application No. 63/286,149, filed on Dec. 6, 2021.

(51) Int. Cl. A61N 1/36; A61N 1/05

(52) U.S. Cl. CPC A61N 1/36038; A61N 1/0541

(57) ABSTRACT

Devices and related methods for providing an electrophonic microphone are described herein. An example method includes recording, using at least one electrode, an early auditory potential, where the at least one electrode is disposed inside a subject's ear in proximity to one of the tympanic membrane, the cochlear promontory, or the round window. The method also includes processing, using a processor, the early auditory potential to generate an audio signal, and transmitting the audio signal to a receiver circuit.

### CROSS-REFERENCE TO RELATED APPLICATIONS

This application claims the benefit of U.S. provisional patent application No. 63/286,149, filed on Dec. 6, 2021, and titled “DEVICES FOR PROVIDING ELECTROPHONIC MICROPHONE AND RELATED METHODS,” the disclosure of which is expressly incorporated herein by reference in its entirety.

### BACKGROUND

Modern hearing devices such as cochlear implants are semi-implantable devices where the implantable portion consists of the receiver/stimulator and the electrode array that is typically inserted into the cochlea. The external components consist of a microphone, speech processor and radiofrequency transmitter coil that allows communication with the receiver/stimulator through the intact skin. It would be desirable to provide a fully implantable hearing device.

### SUMMARY

An example method for providing an electrophonic microphone is described herein. The method includes recording, using at least one electrode, an early auditory potential, where the at least one electrode is disposed inside a subject's ear in proximity to one of the tympanic membrane, the cochlear promontory, or the round window. The method also includes processing, using a processor, the early auditory potential to generate an audio signal, and transmitting the audio signal to a receiver circuit.

In some implementations, the early auditory potential is recorded by the at least one electrode disposed inside the subject's ear in proximity to one of the tympanic membrane, the cochlear promontory, or the round window.

Alternatively or additionally, the early auditory potential includes at least one of a cochlear microphonic (CM), a compound action potential (CAP), a summating potential (SP), or an auditory nerve neurophonic (ANN).

Alternatively or additionally, the method optionally includes implanting the at least one electrode inside the subject's ear.

Alternatively or additionally, the receiver circuit is a component of an auditory prosthetic device, a consumer electronic device, or a covert listening device.

An example device for providing an electrophonic microphone is described herein. The device includes at least one electrode that is configured for implantation inside a subject's ear in proximity to one of the tympanic membrane, the cochlear promontory, or the round window; and a processor coupled to the at least one electrode. The processor is configured to: receive the early auditory potential recorded by the at least one electrode; process the early auditory potential to generate an audio signal; and transmit the audio signal to a receiver circuit.

In some implementations, the device is an auditory prosthetic device, a consumer electronic device, or a covert listening device.

Another example method for providing an electrophonic microphone is described herein. The method includes recording, using at least one electrode, an early auditory potential, where the at least one electrode is disposed, at least partially, outside of a subject's middle ear. The method also includes processing, using a processor, the early auditory potential to generate an audio signal, and transmitting the audio signal to a receiver circuit.

In some implementations, the early auditory potential is recorded by the at least one electrode disposed, at least partially, outside of the subject's middle ear.

Alternatively or additionally, the early auditory potential includes at least one of a cochlear microphonic (CM), a compound action potential (CAP), a summating potential (SP), or an auditory nerve neurophonic (ANN).

Alternatively or additionally, the method optionally includes inserting the at least one electrode into the subject's ear.

Alternatively or additionally, the receiver circuit is a component of an auditory prosthetic device, a consumer electronic device, or a covert listening device.

An example device for providing an electrophonic microphone is described herein. The device includes at least one electrode that is configured for placement, at least partially, outside of a subject's middle ear; and a processor coupled to the at least one electrode. The processor is configured to: receive the early auditory potential recorded by the at least one electrode; process the early auditory potential to generate an audio signal; and transmit the audio signal to a receiver circuit.

In some implementations, the device is an auditory prosthetic device, a consumer electronic device, or a covert listening device.

Another example method for providing an electrophonic microphone is described herein. The method includes recording, using at least one electrode, an early auditory potential, wherein the at least one electrode is disposed, at least partially, within a subject's inner ear. The method also includes processing, using a processor, the early auditory potential to generate an audio signal, and transmitting the audio signal to a receiver circuit.

In some implementations, the early auditory potential is recorded by the at least one electrode is disposed, at least partially, within the subject's inner ear.

In some implementations, the at least one electrode is disposed, at least partially, within the subject's cochlea.

Alternatively or additionally, the receiver circuit is a component of an auditory prosthetic device, a consumer electronic device, or a covert listening device.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example electrophonic microphone environment according to an implementation described herein.

FIG. 2A is an illustration of the anatomy of the human ear by Lars Chittka; Axel Brockmann-Perception Space—The Final Frontier, A PLOS Biology Vol. 3, No. 4, e137 doi: 10.1371/journal.pbio.0030137 (FIG. 1A/Large version), vectorised by Inductiveload, CC BY 2.5.

FIG. 2B is an illustration of the anatomy of the human middle ear by BruceBlaus, Blausen.com staff (2014). “Medical gallery of Blausen Medical 2014”. WikiJournal of Medicine 1 (2).

FIG. 3 are graphs illustrating single-sweep (unaveraged) electrocochleography (ECochG) thresholds by stimulation frequency from a group of individuals with varying degrees of hearing loss.

FIG. 4 are graphs illustrating example recordings from a patient with conductive hearing loss recorded on the promontory in response to condensations and rarefaction stimuli at 500 Hz (top) and 2000 Hz (bottom).

FIG. 5 are graphs illustrating comparison of the different recording sites showing strong correlations between the promontory, round window, inside the round window (scala tympani), and in the cochlear apex.

FIG. 6 is an example computing device.

FIG. 7 is a block diagram illustrating a device for providing an electrophonic microphone according to an implementation described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

Devices for providing an electrophonic microphone and related methods are described herein. In one implementation described herein, an example device includes an implantable microphone, power source and speech processor and a receiver stimulator, e.g., for implantable hearing aid or cochlear implant applications. Instead of using a completely re-designed device, the example device uses an implantable device add-on that interfaces with the existing receiver/stimulator. Specifically, a power source, for example, can be a part of the receiver stimulator, implanted either near the receiver stimulator or at a distant site such as the chest or neck. The speech processor can align with the receiver/stimulator antenna under the skin to communicate with the internal device. Finally, the speech processor can create an internal short between the electrode array and ground electrodes (either on the case or a secondary electrode) that allows access to one of more of the internal electrodes to be used as an electrocochleography (ECochG microphone), obviating the need for a subcutaneous or direct connect ossicular microphone. Thus, the connection is internal-rather than through the intact skin

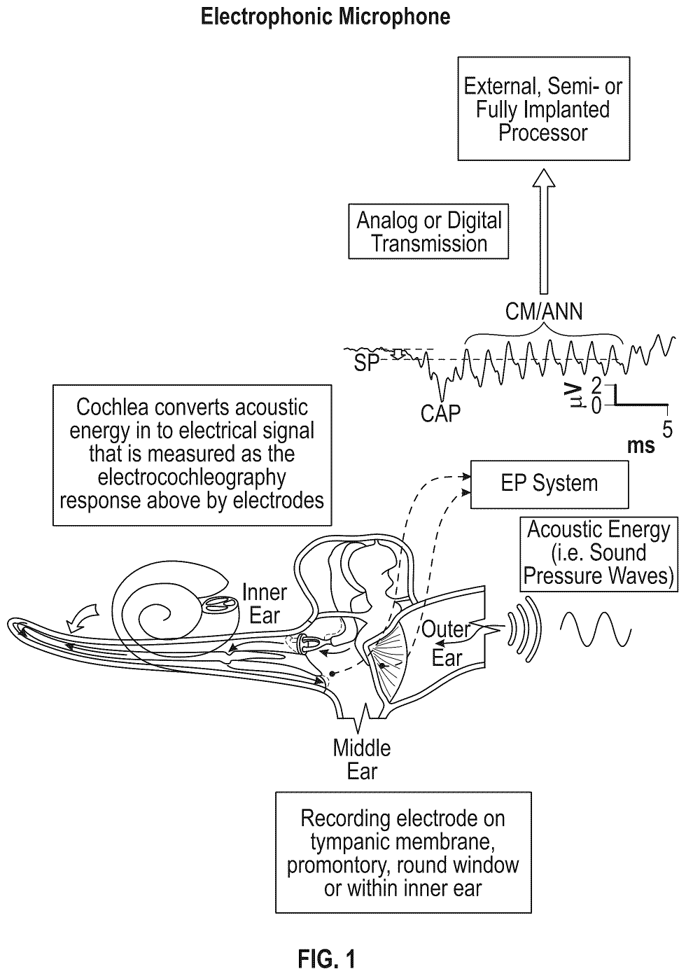

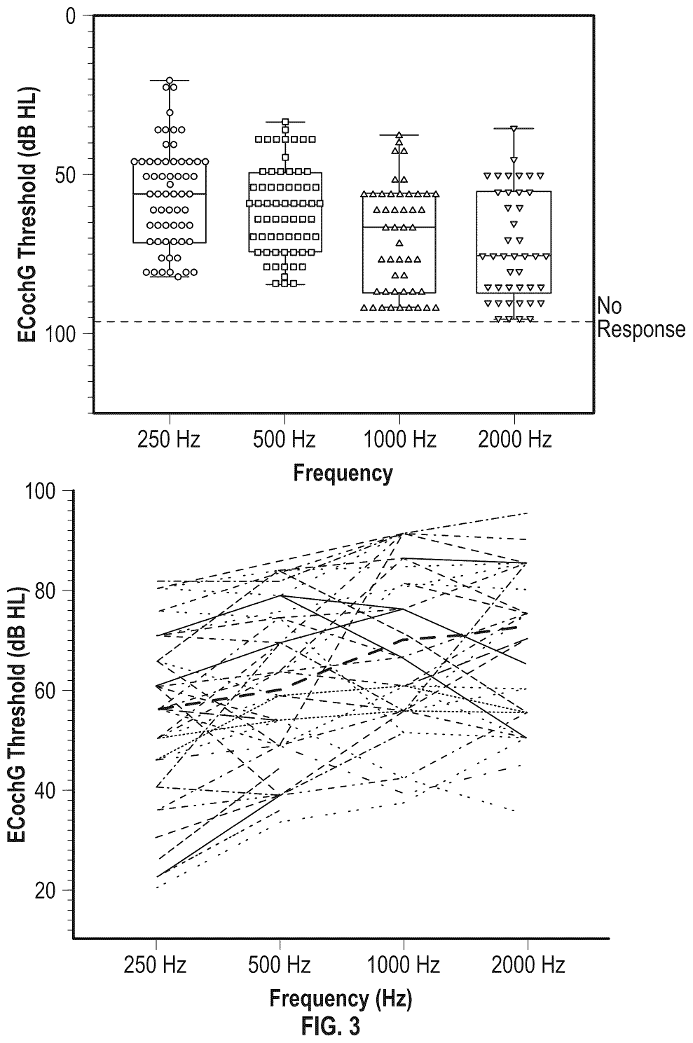

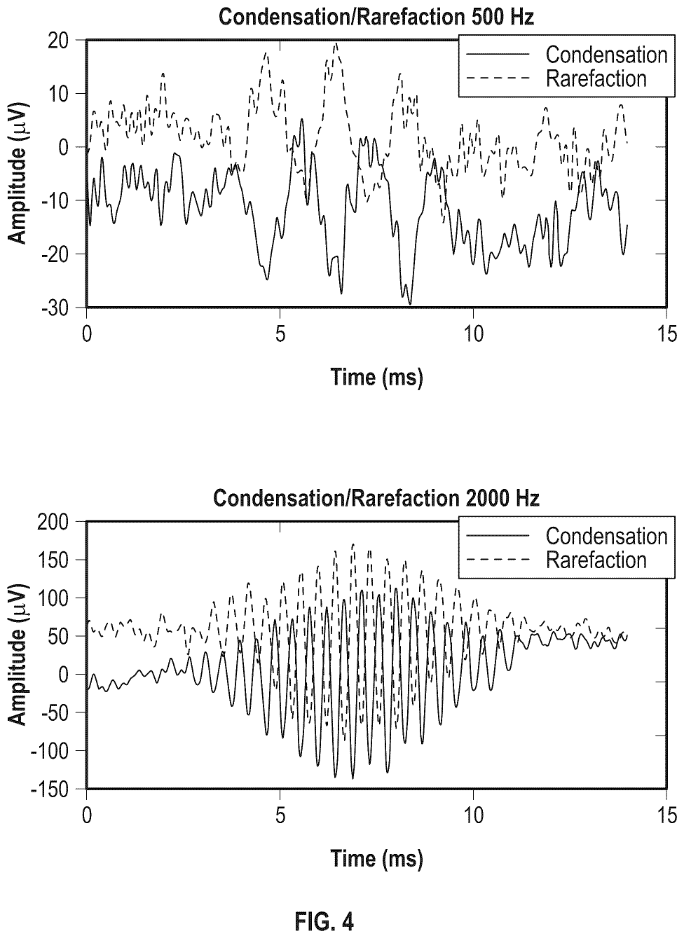

Referring now to FIGS. 1-5, the devices described herein can be implantable, semi-implantable, or wearable. This disclosure contemplates that the devices may include an auditory prosthetic device (e.g., a hearing aid, cochlear implant, or other implantable middle or inner ear stimulator), a consumer electronic device (e.g., an implantable mobile or cellular telephone), or a covert listening device (e.g., a spy device such as a bug). It should be understood that the above devices are provided only as examples. This disclosure contemplates that the devices described herein can be other types of devices including, but not limited to, an auditory assistance device (e.g., a device used by a subject without hearing impairment to improve hearing, for example, in environments with loud background noise such as experienced by machinery operators, vehicle drivers, or pilots). Alternatively, this disclosure contemplates that the devices may be used in conjunction with an auditory prosthetic device, a consumer electronic device, a covert listening device, an auditory assistance device, etc.

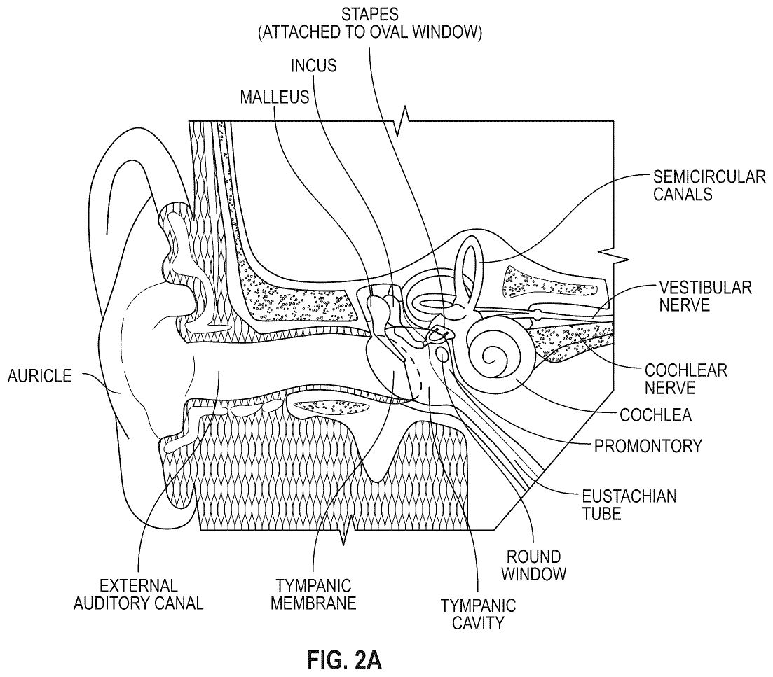

As described herein, the devices are configured to record, using at least one electrode, early auditory potential, which include but are not limited to, a cochlear microphonic (CM), a compound action potential (CAP), a summating potential (SP), or an auditory nerve neurophonic (ANN). Optionally, the at least one electrode is a single electrode or multiple electrodes (e.g., a pair of electrodes, an electrode array, etc.). In some implementations, the at least one electrode is implanted inside the subject's ear, for example, in the middle ear region. Optionally, the at least one electrode is implanted in proximity to one of the tympanic membrane, the cochlear promontory, the round window, the eustachian tube, the epitympanum, the hypotympanum, the mastoid, or nearby these locations. In some implementations, the at least one electrode is placed inside the subject's ear, for example, in the outer ear region (i.e., outside of the middle ear region). Optionally, the at least one electrode is placed in the external ear canal. In these implementations, the at least one electrode may be a skin electrode. In some implementations, the at least one electrode is disposed, at least partially, within the subject's inner ear region, for example, the cochlea. FIGS. 2A-2B illustrate the anatomy of a human's ear.

Auditory prosthetic devices using early auditory potentials recorded in the inner ear region as a microphone are described in detail in WO2021/007412, the disclosure of which is incorporated herein by reference in its entirety. As noted above, in some implementations, the devices described herein use early auditory potentials recorded outside the inner ear region, e.g., in the middle and/or outer ear regions (e.g., external auditory canal, tympanic membrane, promontory, or round window. In other implementations, the devices described herein use early auditory potentials recorded inside the inner ear region (e.g. inside the cochlea or other inner ear region) for applications such as hearing aids, consumer electronic devices, covert listening devices, etc.

In some implementations described herein, the devices are implantable or wearable and can be used either alone or in conjunction with other non-, semi-, or fully implantable devices that use naturally occurring early auditory potentials, mainly but not limited to electrocochleographic (ECochG) responses including the cochlear microphonic and others, as a microphone. This signal can be used as a microphone for sound input to be used in different ways. Possible utilizations include a microphone for hearing aids, implantable auditory devices (cochlear implant or implantable middle ear stimulator), consumer electronics including cell phones, and military-based applications including covert/spy/surveillance technology (e.g. bug).

For the implanted applications, the device is to be placed within the middle ear or in close proximity to the structures of the inner ear. Electrode placement can include, but is not limited to, on the promontory, round window or within the inner ear. The subsequently recorded signal (cochlear microphonic, summating potential, compound action potential, auditory nerve neurophonic) can then be sent either via a wire or wirelessly to other components of the set-up for further processing, transmission, etc.

Such a device includes a recording electrode to record the biologic signal as well as a optionally a signal decoder and components to transmit for further use (wired or wireless). The components can optionally include a power source, especially when using a wireless application. Targets for the microphone signal would be devices as outlined above including hearing aids, cochlear implants, etc.

For the wearable applications, the set-up is similar with the exception that the device is located either be partially or completely outside of the middle ear. Placements include, but are not limited to, the external ear canal or on the tympanic membrane. Additionally, the device can optionally include special skin electrodes on the surface or under the surface that would allow recording of the target signal.

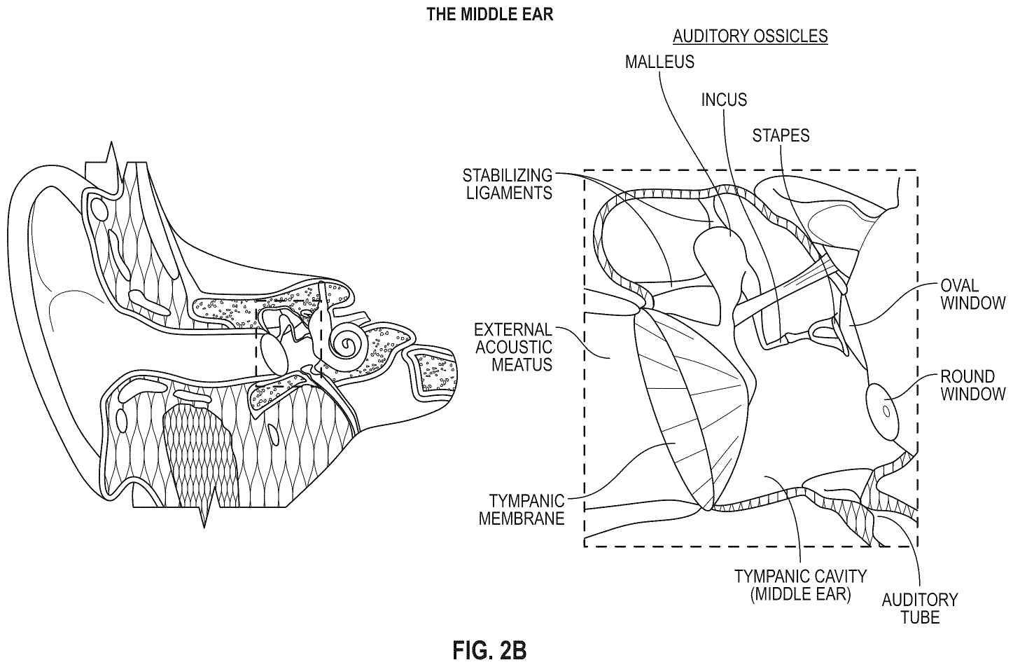

As shown in FIG. 3-5, preliminary data show that ECochG recording sites in the middle ear generate very similar signal amplitudes to those obtained on the round window or within the inner ear (see example). Recording sites outside of the middle ear are somewhat attenuated but can still be used with ears with normal hearing or varying levels of hearing loss. Sound is picked up and signal is sent either as an analogue or digital signal to end device.

An example method for providing an electrophonic microphone is described herein. The method includes recording, using at least one electrode, an early auditory potential. The at least one electrode is disposed near or within the middle ear region, for example, in proximity to structures such as the tympanic membrane, the cochlear promontory (e.g., the rounded hollow prominence formed by the projection outward of the first turn of the cochlea, which may also be referred to as the “promontory of the tympanic cavity”), or the round window or inside the inner ear. FIGS. 2A-2B are provided to illustrate the anatomy of a human's ear. The method also includes processing, using a processor (optionally in some implementations a digital signal processor (DSP)), the early auditory potential to generate an audio signal. As described herein, the audio signal can be used as a microphone for sound input (e.g., a microphone for hearing aids, cochlear implant, consumer electronic devices, covert/spy/surveillance technologies, etc.). The method further includes transmitting the audio signal to a receiver circuit. Such receiver circuit may be a component of an auditory prosthetic device, an auditory assistance device, a consumer electronic device, or a covert listening device. Optionally, the receiver circuit is implanted in the subject's body. Optionally, the receiver circuit is located outside of the subject's body. In some implementation, the receiver circuit transfers the audio signal, for example, to a speaker (e.g., in hearing aid, hearing assistance device, consumer electronic device applications). In other implementations, the receiver circuit is configured to convert the processed audio signal into a stimulation signal, which is transmitted to a stimulation electrode array arranged within the subject's cochlea (e.g., in cochlear implant applications) or an implantable middle ear stimulator such as an ossicular driver. Alternatively or additionally, the receiver circuit transmits the audio signal for storage in memory or data storage.

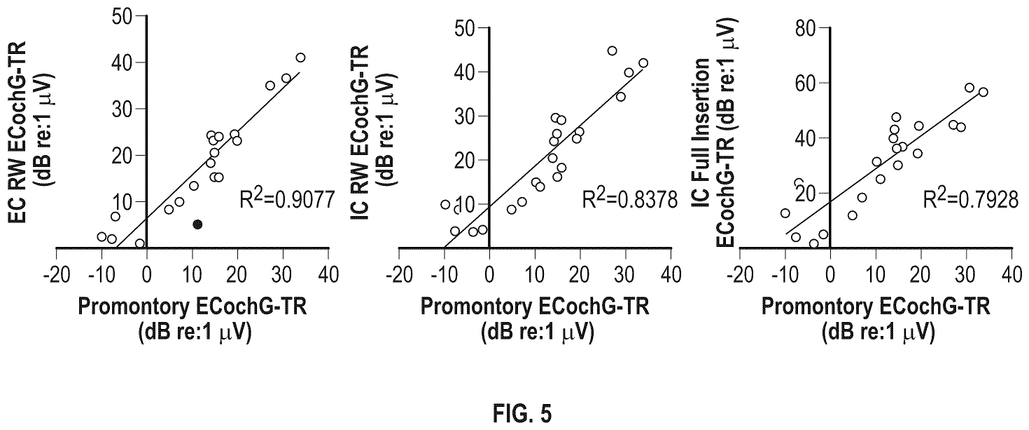

In some implementations, the processor and receiver circuit are parts of the same component. In other implementations, the processor and the receiver circuit are separate components. This disclosure contemplates that the processor can be a processor as described with regard to FIG. 6. As noted above, the processor can optionally be a DSP, which is a specialized microprocessor (e.g., including at least a processor and memory as described with regard to FIG. 6) for signal processing. Signal processing can include, but is not limited to, analog-to-digital conversion (ADC), filtering, compression, etc. of analog signals such as the early auditory potential (e.g., including CM) recorded by the at least one electrode. DSPs are known in the art and are therefore not described in further detail herein. The at least one electrode and the processor can be coupled by a communication link. This disclosure contemplates the communication link is any suitable communication link. For example, a communication link may be implemented by any medium that facilitates signal exchange between the at least one electrode and the processor (includes e.g., wired, wireless, optical links). Additionally, the processor and the receiver circuit can be coupled by a communication link. This disclosure contemplates the communication link is any suitable communication link. For example, a communication link may be implemented by any medium that facilitates signal exchange between the processor and the receiver circuit (includes e.g., wired, wireless, optical links).

In some implementations, the early auditory potential is recorded by the at least one electrode disposed inside the subject's ear in proximity to one of the tympanic membrane, the cochlear promontory, or the round window.

Alternatively or additionally, the method optionally includes implanting the at least one electrode inside the subject's ear.

Another example method for providing an electrophonic microphone is described herein. The method includes recording, using at least one electrode, an early auditory potential. The at least one electrode is disposed (partially or fully) outside of the subject's middle ear, for example, in the subject's external ear canal. FIGS. 2A-2B are provided to illustrate the anatomy of a human's ear. The method also includes processing, using a processor (e.g., optionally a DSP), the early auditory potential to generate an audio signal. As described herein, the audio signal can be used as a microphone for sound input (e.g., a microphone for hearing aids, consumer electronic devices, covert/spy/surveillance technologies, etc.). The method further includes transmitting the audio signal to a receiver circuit. Such receiver circuit may be a component of an auditory prosthetic device, an auditory assistance device, a consumer electronic device, or a covert listening device. Optionally, the receiver circuit is implanted in the subject's body. Optionally, the receiver circuit is located outside of the subject's body. In some implementation, the receiver circuit transfers the audio signal, for example, to a speaker (e.g., in hearing aid, hearing assistance device, consumer electronic device applications). In other implementations, the receiver circuit is configured to convert the processed audio signal into a stimulation signal, which is transmitted to a stimulation electrode array arranged within the subject's cochlea (e.g., in cochlear implant applications) or implantable middle ear stimulator (e.g., ossicular driver). Alternatively or additionally, the receiver circuit transmits the audio signal for storage in memory or data storage.

In some implementations, the early auditory potential is recorded by the at least one electrode disposed, at least partially, outside of the subject's middle ear.

Alternatively or additionally, the method optionally includes inserting the at least one electrode into the subject's ear.

Another example method for providing an electrophonic microphone is described herein. The method includes recording, using at least one electrode, an early auditory potential. The at least one electrode is disposed, at least partially, within a subject's inner ear, for example, in the cochlea. FIGS. 2A-2B are provided to illustrate the anatomy of a human's ear. The method also includes processing, using a processor (e.g., optionally a DSP), the early auditory potential to generate an audio signal. As described herein, the audio signal can be used as a microphone for sound input (e.g., a microphone for hearing aids, consumer electronic devices, covert/spy/surveillance technologies, etc.). The method further includes transmitting the audio signal to a receiver circuit. Such receiver circuit may be a component of an auditory prosthetic device, an auditory assistance device, a consumer electronic device, or a covert listening device. Optionally, the receiver circuit is implanted in the subject's body. Optionally, the receiver circuit is located outside of the subject's body. In some implementation, the receiver circuit transfers the audio signal, for example, to a speaker (e.g., in hearing aid, hearing assistance device, consumer electronic device applications). In other implementations, the receiver circuit is configured to convert the processed audio signal into a stimulation signal, which is transmitted to a stimulation electrode array arranged within the subject's cochlea (e.g., in cochlear implant applications). Alternatively or additionally, the receiver circuit transmits the audio signal for storage in memory or data storage.

In some implementations, the early auditory potential is recorded by the at least one electrode is disposed, at least partially, within the subject's inner ear.

Alternatively or additionally, the method optionally includes inserting the at least one electrode into the subject's inner ear.

It should be appreciated that the logical operations described herein with respect to the various figures may be implemented (1) as a sequence of computer implemented acts or program modules (i.e., software) running on a computing device (e.g., the computing device described in FIG. 6), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 6, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 6 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

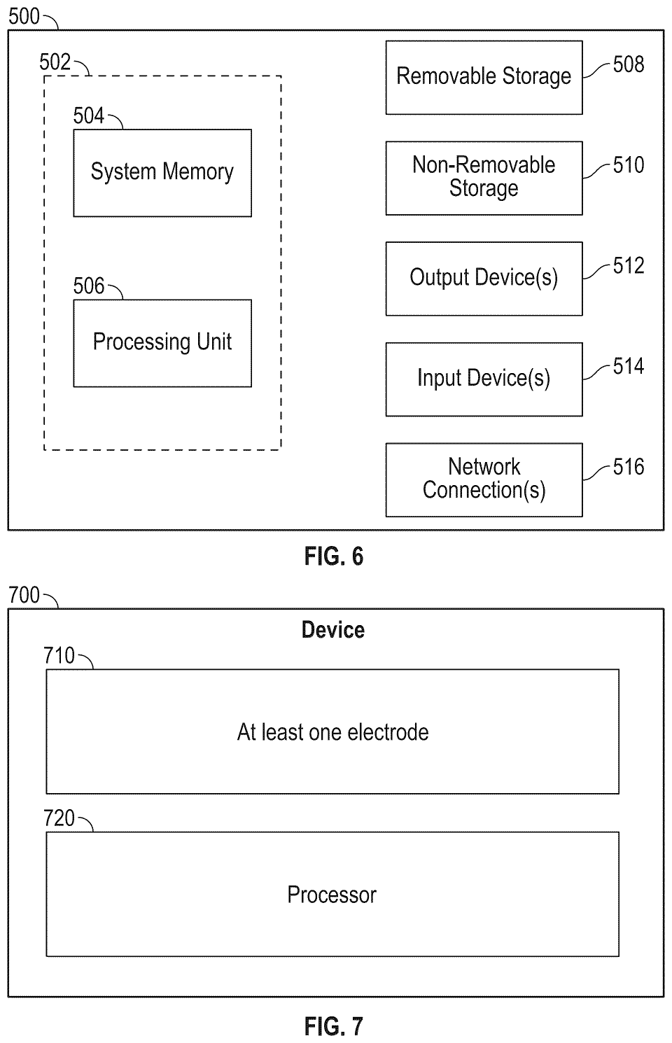

Referring now to FIG. 7, an example device 700 for providing an electrophonic microphone is described. The device 700 can include at least one electrode 710 and a processor 720 operably coupled to the at least one electrode 710. Additionally, the at least one electrode 710 and the processor 720 can be coupled by a communication link. This disclosure contemplates the communication link is any suitable communication link. For example, a communication link may be implemented by any medium that facilitates signal exchange between the at least one electrode 710 and processor 720. As described herein, the device 700 can be an auditory prosthetic device, a consumer electronic device, or a covert listening device. It should be understood that auditory prosthetic devices, consumer electronic devices, and covert listening devices are provided only as examples.

In some implementations described herein, the at least one electrode 710 is configured for implantation inside a subject's ear in proximity to one of the tympanic membrane, the cochlear promontory, or the round window. In other implementations described herein, the at least one electrode 710 is configured for placement, at least partially, outside of a subject's middle ear. In yet other implementations described herein, the at least one electrode 710 is configured for placement, at least partially, within a subject's inner ear.

Optionally, in some implementations, the at least one electrode 710 can include a plurality of electrodes. In some implementations, the early auditory potential can be recorded at one or more of the electrodes of the electrode array. In other implementations, the early auditory potential can be recorded at each of the electrodes of the electrode array (i.e., all of the electrodes of the electrode array). As described herein, the early auditory potential can be processed by the processor 720 to generate an audio signal.

Optionally, in some implementations, the processor 720 is a digital signal processor (DSP). A DSP is a specialized microprocessor (e.g., including at least a processor and memory as described with regard to FIG. 6) for signal processing. Signal processing can include, but is not limited to, analog-to-digital conversion (ADC), filtering, compression, etc. of analog signals such as the early auditory potential recorded by the at least one electrode 710.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for providing an electrophonic microphone, comprising:
recording, using at least one electrode, an early auditory potential, wherein the at least one electrode is disposed inside a subject's ear in proximity to one of the tympanic membrane, the cochlear promontory, or the round window;
processing, using a processor, the early auditory potential to generate an audio signal; and
transmitting the audio signal to a receiver circuit.

2. The method of claim 1, wherein the early auditory potential is recorded by the at least one electrode disposed inside the subject's ear in proximity to one of the tympanic membrane, the cochlear promontory, or the round window.

3. The method of claim 1, wherein the early auditory potential comprises at least one of a cochlear microphonic (CM), a compound action potential (CAP), a summating potential (SP), or an auditory nerve neurophonic (ANN).

4. The method of claim 1, further comprising implanting the at least one electrode inside the subject's ear.

5. The method of claim 1, wherein the receiver circuit is a component of an auditory prosthetic device, an auditory assistance device, a consumer electronic device, or a covert listening device.

6. A device, comprising:
at least one electrode that is configured for implantation inside a subject's ear in proximity to one of the tympanic membrane, the cochlear promontory, or the round window; and
a processor coupled to the at least one electrode, wherein the processor is configured to:
receive the early auditory potential recorded by the at least one electrode;
process the early auditory potential to generate an audio signal; and
transmit the audio signal to a receiver circuit.

7. The device of claim 6, wherein the device is an auditory prosthetic device, an auditory assistance device, a consumer electronic device, or a covert listening device.

8. A method for providing an electrophonic microphone, comprising:
recording, using at least one electrode, an early auditory potential, wherein the at least one electrode is disposed, at least partially, outside of a subject's middle ear;
processing, using a processor, the early auditory potential to generate an audio signal; and
transmitting the audio signal to a receiver circuit.

9. The method of claim 8, wherein the early auditory potential is recorded by the at least one electrode disposed, at least partially, outside of the subject's middle ear.

10. The method of claim 8, wherein the at least one electrode is a skin electrode.

11. The method of claim 8, wherein the early auditory potential comprises at least one of a cochlear microphonic (CM), a compound action potential (CAP), a summating potential (SP), or an auditory nerve neurophonic (ANN).

12. The method of claim 8, further comprising inserting the at least one electrode into the subject's ear.

13. The method of claim 8, wherein the receiver circuit is a component of an auditory prosthetic device, an auditory assistance device, a consumer electronic device, or a covert listening device.

14. A device, comprising:
at least one electrode that is configured for placement, at least partially, outside of a subject's middle ear; and
a processor coupled to the at least one electrode, wherein the processor is configured to:
receive the early auditory potential recorded by the at least one electrode;
process the early auditory potential to generate an audio signal; and
transmit the audio signal to a receiver circuit.

15. The device of claim 14, wherein the device is an auditory prosthetic device, an auditory assistance device, a consumer electronic device, or a covert listening device.

16. A method for providing an electrophonic microphone, comprising:
recording, using at least one electrode, an early auditory potential, wherein the at least one electrode is disposed, at least partially, within a subject's inner ear;
processing, using a processor, the early auditory potential to generate an audio signal; and
transmitting the audio signal to a receiver circuit.

17. The method of claim 16, wherein the early auditory potential is recorded by the at least one electrode is disposed, at least partially, within the subject's inner ear.

18. The method of claim 16, wherein the at least one electrode is disposed, at least partially, within the subject's cochlea.

19. The method of claim 16, wherein the early auditory potential comprises at least one of a cochlear microphonic (CM), a compound action potential (CAP), a summating potential (SP), or an auditory nerve neurophonic (ANN).

20. The method of claim 16, wherein the receiver circuit is a component of an auditory prosthetic device, an auditory assistance device, a consumer electronic device, or a covert listening device.
